(19) United States
(12) Patent Application Publication
(10) Pub. No.: US 2020/0324090 A1
(43) Pub. Date: Oct. 15, 2020

(54) DEPLOYMENT SYSTEM ACCESS SHEATH

(71) Applicant: W. L. Gore & Associates, Inc., Newark, DE (US)

(72) Inventors: James R. Black, Flagstaff, AZ (US); Jacob T. Earl, Flagstaff, AZ (US); Nathan P. Dahl, Flagstaff, AZ (US); Nathan L. Friedman, Flagstaff, AZ (US)

(21) Appl. No.: 16/845,256

(22) Filed: Apr. 10, 2020

Related U.S. Application Data
(60) Provisional application No. 62/845,068, filed on May 8, 2019.
(60) Provisional application No. 62/832,235, filed on Apr. 10, 2019.

(51) Int. Cl. A61M 25/09; A61M 25/01

(52) U.S. Cl. CPC A61M 25/09; A61M 2025/09008; A61M 2025/0161; A61M 25/0147

(57) ABSTRACT

Various aspects of the present disclosure are directed toward apparatuses, systems, and methods that include an access sheath including an elongate body with an internal lumen to facilitate delivery of a device to a target location within a patient. The elongate body may include a distal portion having a distal opening and a plurality of curved segments configured to orient the distal opening relative to the target location.

### CROSS-REFERENCE TO RELATED APPLICATION

This application claims the benefit of Provisional Application No. 62/845,068, filed May 8, 2019, and also claims the benefit of Provisional Application No. 62/832,235, filed Apr. 10, 2019, both of which are incorporated herein by reference in their entireties for all purposes.

### FIELD

This disclosure relates to delivery sheaths and methods. More particularly, the disclosure relates to dilator and catheter introducer systems and methods for traversing blood vessels and organs.

### BACKGROUND

Catheter introducer systems are often introduced into blood vessels and organs for intraluminal diagnostics, treatment and delivery of medical devices and structures. The type of catheter introducer system utilized depends on the medical procedure performed, the route in the body taken, and individual patient anatomy, among other factors.

### SUMMARY

According to one example (“Example 1”), a delivery system includes an access sheath including an elongate body with an internal lumen to facilitate delivery of a device to a target location within a patient, the elongate body extending in a direction of extension and including: a proximal portion extending in a first plane, and a distal portion having a distal opening and a plurality of curved segments configured to orient the distal opening relative to the target location, the distal portion including, a proximal curved segment extending within in a first plane to define a proximal nominal angular offset in the direction of extension of the elongate body, and a distal curved segment extending within a second plane to define a distal nominal angular offset in the direction of extension of the elongate body, the first and second planes being angularly offset from one another.

According to another example (“Example 2”), further to the system of Example 1, the proximal nominal angular offset is between approximately 65 and 75 degrees and the distal nominal angular offset is between approximately 25 and 35 degrees.

According to another example (“Example 3”), further to the system of Example 2, the proximal nominal angular offset is approximately 70 degrees and the distal nominal angular offset is approximately 30 degrees.

According to another example (“Example 4”), further to the system of any one of Examples 1-3, the proximal curved segment and the distal curved segment are in different planes and offset between approximately 80-110 degrees.

According to another example (“Example 5”), further to the system of any one of Examples 1-4, the proximal curved segment is an x-z plane the distal curved segment is in a y-z plane.

According to another example (“Example 6”), further to the system of any one of Examples 1-5, the system also includes a catheter configured to pass through the internal lumen of the elongate body and the elongate body is configured to maintain the proximal nominal angular offset and the distal nominal angular offset when the catheter is within the distal portion of the elongate body.

According to another example (“Example 7”), further to the system of Example 6, the catheter facilitates delivery of an implantable medical device.

According to another example (“Example 8”), further to the system of Example 7, the implantable medical device is selected from a group consisting of: a left atrial appendage occluder, an occluder, a stent, a stent-graft, a shunt, a sensor, a pressure sensing device, and a diagnostic device.

According to another example (“Example 9”), further to the system of Example 7, the implantable medical device is a left atrial appendage occluder and the proximal curved segment is configured to guide the elongate body through a septum in heart turn toward a left atrial appendage and the distal curved segment is configured to align the catheter with a longitudinal axis of the left atrial appendage.

According to another example (“Example 10”), further to the system of Example 6, the catheter is selected from a group consisting of a medical device delivery catheter, an ablation catheter a drug delivery catheter, and a contrast solution delivery catheter.

According to another example (“Example 11”), further to the system of any one of Examples 1-10, the system also includes a projection coupled to the proximal portion of the elongate body configured to indicate a direction of the proximal curved segment.

According to another example (“Example 12”), further to the system of Example 11, the projection is a port coupled to the proximal portion of the elongate body, and the port extends in a direction relative to the elongate body in a common direction as the proximal curved segment.

According to another example (“Example 13”), further to the system of any one of Examples 1-12, the system also includes a proximal steering element configured to actuate the proximal curved segment in-plane.

According to another example (“Example 14”), further to the system of Example 13, the proximal steering element is a steering wire coupled to the proximal curved segment configured to actuate the proximal curved segment in response to a force applied to the steering wire.

According to another example (“Example 15”), further to the system of Example 13, proximal steering element includes a series of articulating structures arranged about the proximal curved segment and configured to individually actuate to effect curvature of the proximal curved segment.

According to another example (“Example 16”), further to the system of any one of Examples 1-15, the system also includes a distal steering element configured to rotate the distal curved segment.

According to another example (“Example 17”), further to the system of Example 16, the distal steering element is a steering wire coupled to the distal curved segment configured to rotate the distal curved segment in response to a force applied to the steering wire.

According to another example (“Example 18”), further to the system of any one of Examples 1-15, the system also includes a distal steering element configured to deflect the distal curved segment about a 360 degree range.

According to another example (“Example 19”), further to the system of Example 18, the distal steering element is a steering wire coupled to the distal curved segment configured to deflect the distal curved segment in response to a force applied to the steering wire.

According to another example (“Example 20”), further to the system of Example 18, the distal steering element includes a series of articulating structures arranged about the distal curved segment and configured to individually actuate to effect deflection of the distal curved segment.

According to another example (“Example 21”), further to the system of Example 13, the distal steering element includes a series of articulating structures arranged about the distal curved segment and configured to individually actuate to effect rotation of the distal curved segment.

According to another example (“Example 22”), further to the system of any one of Examples 13-21, the access sheath includes the proximal steering element and the distal steering element.

According to one example (“Example 23”), a method of delivering an implantable medical device to a target location within a patient includes arranging an access sheath within a patient, the access sheath including an elongate body with an internal lumen, a proximal portion extending in a first plane, and a distal portion having a distal opening and a plurality of curved segments; and orienting the distal opening of the access sheath relative to the target location using a proximal curved segment of the distal portion extending within in a first plane to define a proximal nominal angular offset in the direction of extension of the elongate body and a distal curved segment of the distal portion extending within a second plane to define a distal nominal angular offset in the direction of extension of the elongate body, the first and second planes being angularly offset from one another.

According to another example (“Example 24”), further to the method of Example 23, the method also includes arranging a delivery catheter through the internal lumen of the access sheath and delivering an implantable medical device arranged on the delivery catheter through the distal opening of the access sheath to the target location.

According to another example (“Example 25”), further to the method of Example 24, the implantable medical device is a left atrial appendage occluder and the proximal curved segment is configured to guide the elongate body through a septum in heart turn toward a left atrial appendage and the distal curved segment is configured to align the catheter with a longitudinal axis of the left atrial appendage.

According to one example (“Example 26”), a delivery system includes an access sheath including an elongate body with an internal lumen to facilitate delivery of a device to a target location within a patient, the elongate body extending in a direction of extension and including: a proximal portion extending in a first plane, and a distal portion having a distal opening and a plurality of segments configured to orient the distal opening relative to the target location, a proximal steering element configured to deflect a proximal one of the plurality of segments to extend within in a first plane to define a proximal nominal angular offset in the direction of extension of the elongate body, and a distal steering element configured to deflect a distal one of the plurality of segments within a second plane to define a distal nominal angular offset in the direction of extension of the elongate body.

According to another example (“Example 27”), further to the system of Example 26, the proximal nominal angular offset is between approximately 65 and 75 degrees and the distal nominal angular offset is between approximately 25 and 35 degrees.

According to another example (“Example 28”), further to the system of Example 27, the proximal nominal angular offset is approximately 70 degrees and the distal nominal angular offset is approximately 30 degrees.

According to another example (“Example 29”), further to the system of Example 26, at least one of the proximal one of the plurality of segments and the distal one of the plurality of segments is substantially aligned with the direction of extension of the elongate body prior to deflection.

According to another example (“Example 30”), further to the system of Example 26, the proximal one of the plurality of segments is a proximal curved segment prior to deflection.

According to another example (“Example 31”), further to the system of Example 30, the distal one of the plurality of segments is a distal curved segment prior to deflection.

According to another example (“Example 32”), further to the system of Example 31, the proximal steering element and the distal steering element and configured to deflect the proximal curved segment and the distal curved segment to different planes offset between approximately 80-110 degrees.

According to another example (“Example 33”), further to the system of any one of Examples 26-33, the first and second planes being angularly offset from one another.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a side view of an example delivery system for accessing a blood vessel, in accordance with various aspects of the present disclosure;

FIG. 2 is a perspective view of a distal end portion of an example access sheath that may be used with a delivery system, in accordance with various aspects of the present disclosure;

FIG. 3 is a view of the distal end portion of the access sheath, shown in FIG. 2, in a first plane, in accordance with various aspects of the present disclosure;

FIG. 4 is a view of the distal end portion of the access sheath h, shown in FIGS. 2-3, in a second plane, in accordance with various aspects of the present disclosure;

FIG. 5 is a view of the distal end portion of the access sheath, shown in FIGS. 2-4, in a third plane, in accordance with various aspects of the present disclosure;

FIG. 6 is another perspective view of the distal end portion of the access sheath, shown in FIGS. 2-5, in accordance with various aspects of the present disclosure;

FIG. 7 is side view of the access sheath, shown in FIGS. 2-6, in accordance with various aspects of the present disclosure;

FIG. 8 is a cross-sectional view of a human heart in which a delivery system including an example access sheath is positioned in preparation for deployment of an occlusive device into a LAA of the heart, in accordance with various aspects of the present disclosure;

FIG. 9 is a perspective view of a distal end portion of an example access sheath that may be steerable, in accordance with various aspects of the present disclosure; and

FIG. 10 is side view of an another example access sheath in accordance with various aspects of the present disclosure.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error or minor adjustments made to optimize performance, for example. In the event it is determined that individuals having ordinary skill in the relevant arts would not readily ascertain values for such reasonably small differences, the terms “about” and “approximately” can be understood to mean plus or minus 10% of the stated value.

Certain terminology is used herein for convenience only. For example, words such as “top”, “bottom”, “upper,” “lower,” “left,” “right,” “horizontal,” “vertical,” “upward,” and “downward” merely describe the configuration shown in the figures or the orientation of a part in the installed position. Indeed, the referenced components may be oriented in any direction. Similarly, throughout this disclosure, where a process or method is shown or described, the method may be performed in any order or simultaneously, unless it is clear from the context that the method depends on certain actions being performed first.

A coordinate system is presented in the Figures and referenced in the description in which the “Y” axis corresponds to a vertical direction, the “X” axis corresponds to a horizontal or lateral direction, and the “Z” axis corresponds to the interior/exterior direction.

### Description of Various Embodiments

Persons skilled in the art will readily appreciate that various aspects of the present disclosure can be realized by any number of methods and apparatus configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

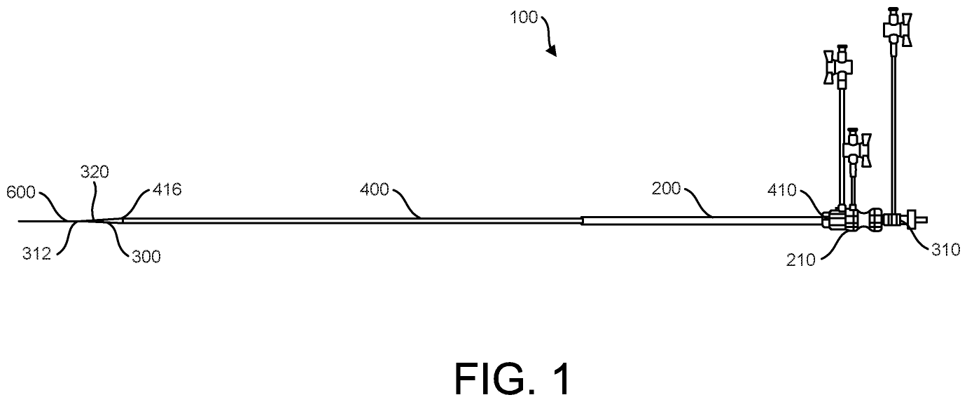

FIG. 1 depicts an example delivery system 100 for accessing a target location within a patient. The delivery system 100 may include a dilator 300 is disposed in an access sheath 400. In certain instances, the delivery system 100 may also include an introducer sheath 200 to facilitate introduction of the access sheath 400 into the patient. The access sheath 400 may be arranged within the introducer sheath 200. In certain instances, the delivery system 100 may also include a guidewire 600 that is used to navigate to a target location within the patient.

A lumen of the introducer sheath 200 may have a diameter that is operable to slidingly receive the access sheath 400 therein and to allow the access sheath 400 to advance within the lumen. For example, in some embodiments, the lumen of the introducer sheath may have an inner diameter of between substantially 1 mm to substantially 10 mm (e.g., between substantially 4 mm to substantially 8 mm). In certain instances, the introducer sheath 200 may be coupled to a hub 210 that can include an inflatable lumen valve and a flush-port equipped hub at or adjacent a proximal end of the introducer sheath 200. The hub 210 may include a lumen valve having an annular seal that is actuatable via a Luer port (e.g., injection of fluid such as saline or air into valve via the Luer port expands the annular seal to close the entirety of lumen of the introducer 200 or a portion thereof and retraction of fluid opens the lumen of the introducer 200).

In certain instances the dilator 300 may be an elongated tubular member that may be used to enlarge or stretch a body part, such as a blood vessel, cavity, canal, or orifice. The dilator 300 may be used to introduce and guide a sheath into and through a blood vessel. In certain instances, the dilator 300 may be relatively less stiff than the access sheath 400. In addition, a dilator lumen may have a diameter of between substantially 0.038 inch to substantially 0.05 inch (e.g., a diameter large enough to receive a 0.035-inch guidewire) or the dilator 300 may not include a lumen.

In certain instances, a distal end 312 of the dilator 300 may include a dilator distal tip 320. The dilator distal tip 320 may be an integral element of the distal end 312 of the dilator 300 or an element that is coupled to the distal end 312 of the dilator 300. The distal end 312 of the dilator 300 may be tapered, but in most embodiments, a taper is provided on the outer diameter of the dilator distal tip 320 so that blood vessels are not exposed to steps and edges.

In certain instances, the dilator 300 may also include a hub 310 coupled or adjacent to a proximal end of the dilator 300. The hub 310 may be configured to couple the dilator 300 to the access sheath 400 (e.g., via keyed features of the hub 310 and of the access sheath 400) to resist separation of the dilator 300 and access sheath 400 and/or to resist relative rotation between the dilator 300 and access sheath 400.

As shown, the access sheath 400 includes a hub 410 having a lumen seal configured to restrict flow out of a proximal end of the access sheath 400 while still permitting the dilator 300 to be inserted and removed through access sheath 400. In certain instances, the lumen seal of the hub 410 may be an elastomeric sheet disposed across a lumen of the access sheath 400 having one or more central slit(s) through which the dilator 300 can be inserted. In certain embodiments, the hub 410 comprises a Luer port through which liquid can be injected into or withdrawn from the lumen of the access sheath 400, such as, for example, to flush the access sheath lumen.

In certain instances, a distal end 416 of the access sheath 400 may include one or more curvatures or changes in direction. The distal end 416 of the access sheath 400 may include curvatures (in a common plane or in different planes) to facilitate treatment of a target location within a patient. The access sheath may be used to route or delivery diagnostic devices, therapeutic devices, or implantable medical device delivery catheters (generally devices) to a target location. These devices, for example, may be arranged through the access sheath 400 to facilitate delivery of a device to a target location within a patient. The distal end 416 of the access sheath 400 may include one or more curvatures to facilitate alignment or delivery of the devices.

The access sheath 400, for example, may facilitate delivery of a left atrial appendage occluder (LAAO) device to occlude a left atrial appendage of a patient. In these instances, the LAAO device may be collapsed to a delivery diameter on a delivery catheter in order to be routed to the left atrial appendage of the patient. The delivery catheter is arranged through the access sheath 400, which has been positioned adjacent the left atrial appendage by way of the guidewire 600. As explained in further detail below, the one or more curvatures of the distal end 416 of the access sheath 400 may align the delivery catheter relative to the left atrial appendage to facilitate a desired deployment. In certain instances, the access sheath 400 is configured to maintain the one or more curvatures when a catheter or device is passed through the lumen of the access sheath 400.

The access sheath 400 may be shaped or curved depending on the device used for treatment or diagnosis. The access sheath 400 may be used for other implantable medical devices such as occluder, stents, stent-grafts, shunts, and can also be used for ablation catheters, sensors, pressure/diagnostics devices, drug delivery systems, contrast solution delivery, or other similar devices. As another example, when used for ablation, the access sheath 400 may include one or more curvatures to align an ablation catheter with a portion of the heart that is to be ablated. As another example, when used for delivery an occluder for an atrial septal defect (ASD), the access sheath 400 may include one more curvatures to align the access sheath 400 (and a delivery catheter carrying the occluder for the atrial septal defect) with the atrial septal defect.

In certain instances, the delivery system 100 may include multiple access sheaths (e.g., 400) each defining a different curved section. For example, a system or kit of the present access sheaths for a particular procedure (e.g., occlusion of an LAAO or repair of an ASD) can include a plurality of access sheaths with respective curved sections that are similar in overall configurations but sized for variations in sizes within a group of patients (e.g., children, adolescent males, adult females, and/or the like). The number and type of access sheaths included in such a kit may depend on an intended procedure (e.g., due to the particular location in a patient's circulatory system at which the procedure is to be performed and/or the path taken within the body to arrive at such a particular location).

In some embodiments, the access sheath 400 may include one or more radiopaque marker(s), such as, for example, an element that is coupled to the access sheath 400 or by integrating one or more doping material(s) (e.g., barium sulfate) into the construct so as to assist with imaging by x-ray techniques, for example. The radiopaque element(s) may be arranged at a beginning or end of one of the curvatures in the distal end 416 of the access sheath 400 to show the location of the one or more curvatures to an operating physician.

Various materials may be used to achieve the properties (e.g., default shape, stiffness, and/or the like) described herein for the access sheath 400. For example, in some embodiments, the access sheath body portion 404 can include a thermoplastic material (e.g., in extruded form) such as nylon or PEBAX™ m Polyether Block Amide copolymer (Arkema, Inc., King of Prussia, Pa.), and/or a metallic material (e.g., in coiled or braided wire form or tubular form) such as stainless steel or nickel titanium alloy (nitinol).

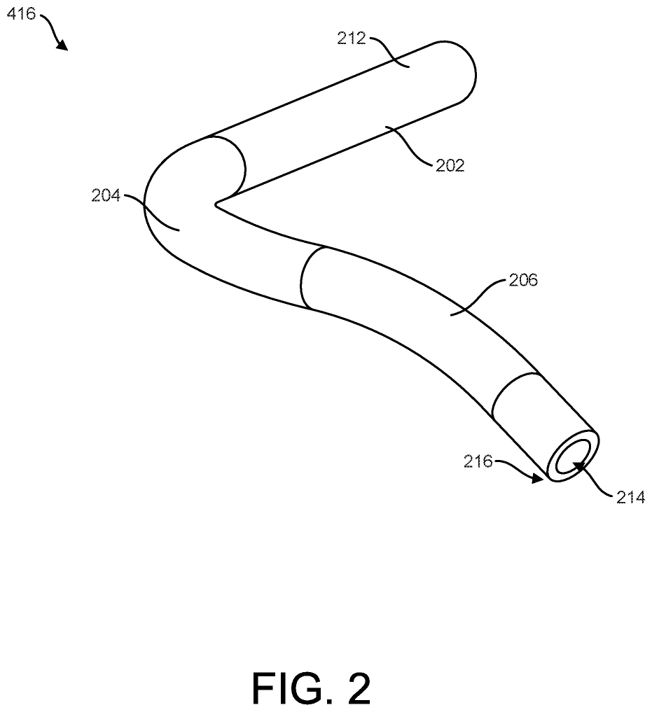

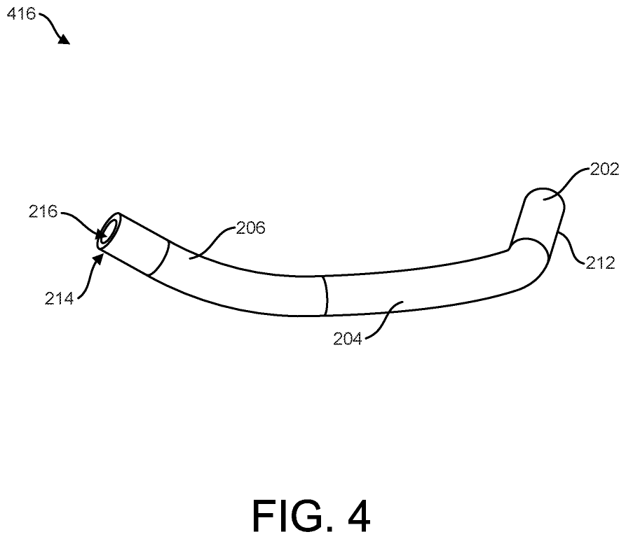

FIG. 2 is a perspective view of a distal portion 416 of an example access sheath 400 that may be used with a delivery system, in accordance with various aspects of the present disclosure. As shown in FIG. 2, the access sheath 400 includes an elongate body 202 with an internal lumen 214 to facilitate delivery of a device to a target location within a patient. The access sheath 400 may include a proximal portion 212 that is at least partially accessible outside of a patient. The proximal portion 212 extends in a direction of extension, which may be a first plane (as is discussed in further detail in FIG. 4).

The distal portion 416 has a distal opening 216 and, as discussed above, the distal portion 416 may include a plurality of curved segments configured to orient the distal opening 216 relative to the target location. In certain instances, the distal portion 418 includes a proximal curved segment 204 defining a proximal nominal angular offset and a distal curved segment 206 extending within a second plane to define a distal nominal angular offset in the direction of extension of the distal portion 416 of the elongate body 202.

In certain instances, the proximal curved segment 204 and the distal curved segment 206 may be curved in different directions. The different directions of curvature may be within the same plane or within different planes but in a common direction relative to another portion of the access sheath 400. For example and as shown in FIG. 2, the proximal curved segment 204 and the distal curved segment 206 generally direct the elongate body 202 in a direction that is different than a direction of extension of the proximal portion 212.

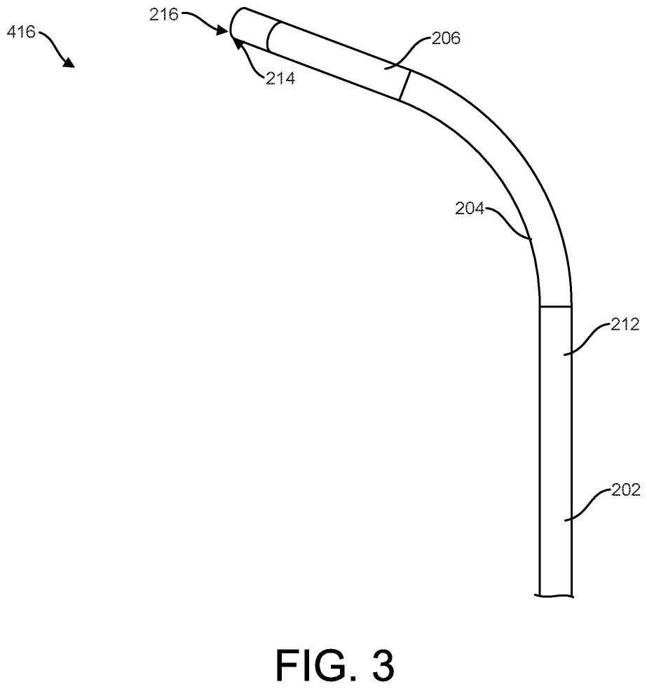

FIG. 3 is a view of the distal end 416 of the access sheath 400, shown in FIG. 2, in a first plane, in accordance with various aspects of the present disclosure. As viewed from in an x-y plane (from a z-direction), the distal end 416 of the access sheath 400 may change direction relative to the proximal portion 212 of the elongate body 202. As shown, each of the proximal curved segment 204 and the distal curved segment 206 curve the elongate body 202 in a common direction as viewed from in an x-y plane (from a z-direction). In other instances, the proximal curved segment 204 and the distal curved segment 206 curve the elongate body 202 in different directions as viewed from in an x-y plane (from a z-direction).

In certain instances, the distal end 416 of the access sheath 400 may include multiple curvatures relative to the proximal portion 212 of the elongate body 202 as viewed from in an x-y plane (from a z-direction). In certain instances, the distal end 416 of the access sheath 400 may change direction multiple times within those multiple curvatures relative to the proximal portion 212 of the elongate body 202 as viewed from in an x-y plane (from a z-direction).

FIG. 4 is a view of the distal end 416 of the access sheath 400, shown in FIGS. 2-3, in a second plane, in accordance with various aspects of the present disclosure. As viewed from in an y-z plane (from the x-direction), the distal end 416 of the access sheath 400 may change direction relative to the proximal portion 212 of the elongate body 202. As shown, each of the proximal curved segment 204 and the distal curved segment 206 curve the elongate body 202 in a common direction as viewed from in an y-z plane (from the x-direction). The proximal curved segment 204 beings the curvature in the elongate body relative to the proximal end 212, and the distal curved segment 206 continues the curvature. In certain instances and as shown, the proximal curved segment 204 and the distal curved segment 206 change direction or curve within the y-z plane. For example and as is shown, the distal curved segment 206 curves the elongate body upwardly within the y-z plane relative to the proximal curved segment 204.

In certain instances, the distal end 416 of the access sheath 400 may include additional curvatures relative to the proximal portion 212 of the elongate body 202 as viewed from in an y-z plane (from the x-direction). In certain instances, the distal end 416 of the access sheath 400 may change direction multiple times within those multiple curvatures relative to the proximal portion 212 of the elongate body 202 as viewed from in an y-z plane (from the x-direction).

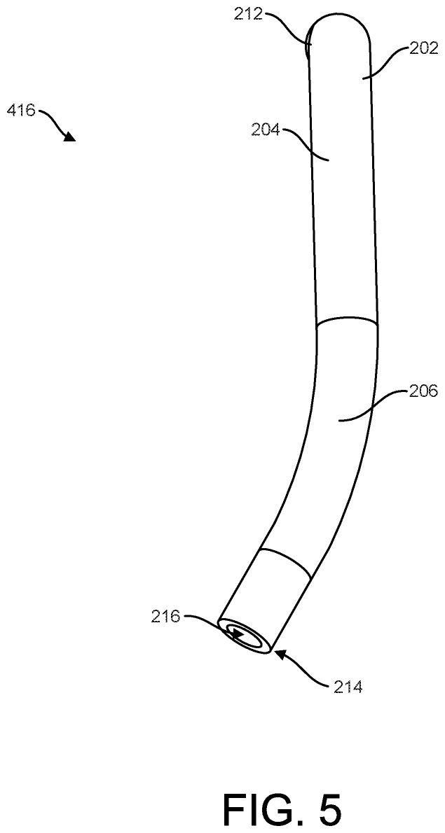

FIG. 5 is a view of the distal end 416 of the access sheath 400, shown in FIGS. 2-4, in a third plane, in accordance with various aspects of the present disclosure. As viewed from in an x-z plane (from the y-direction), the distal end 416 of the access sheath 400 may change direction relative to the proximal portion 212 of the elongate body 202. As shown, the proximal curved segment 204 is relatively un-curved relative to the proximal portion 212 of the elongate body 202 in an x-z plane (from the y-direction). The distal curved segment 206 may curve in a different direction relative to the proximal curved segment 204 is relatively in an x-z plane (from the y-direction).

In certain instances, the distal end 416 of the access sheath 400 may include additional curvatures relative to the proximal portion 212 of the elongate body 202 as viewed from in an x-z plane (from the y-direction). In certain instances, the distal end 416 of the access sheath 400 may change direction multiple times within those multiple curvatures relative to the proximal portion 212 of the elongate body 202 as viewed from in an x-z plane (from the y-direction).

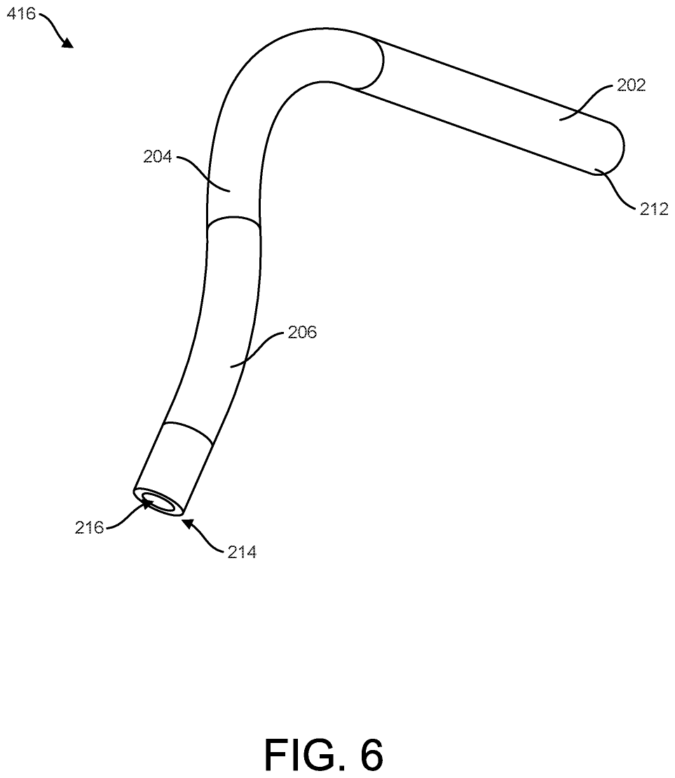

FIG. 6 is another perspective view of the distal end 416 access sheath, shown in FIGS. 2-5, in accordance with various aspects of the present disclosure. As shown in comparing FIGS. 3-5 and in the perspective view of FIGS. 2 and 6, the distal portion 416 of the elongate body 202 may include the proximal curved segment 204 extending within in a first plane to define a proximal nominal angular offset distal portion in the direction of extension of the elongate body 202 and the distal curved segment 206 extending within a second plane to define a distal nominal angular offset in the direction of extension of the elongate body 202. In addition and, the first and second planes may be angularly offset from one another.

The distal portion 416 of the elongate body 202 may include additional curvatures or different curvatures depending on the diagnostic or treatment use of the access sheath 400. In certain instances, the curve(s) of the distal portion 416 may facilitate specific alignment of the access sheath 400 relative to a treatment location. For example and as shown, the access sheath 400 may be used for deployment of a LAAO device. in these instances, the proximal curved segment 204 is configured to guide the access sheath 400 through the septum in the art (and reside in the septum) and turn the access sheath 400 toward the left atrial appendage. In addition, the distal curved segment 206 is configured to align the access sheath 400 with the left atrial appendage. In certain instances, the distal curved segment 206 is configured to align the access sheath 400 with a longitudinal axis of the left atrial appendage. Aligning of the access sheath 400 facilitates deployment of the LAAO device by directing a delivery catheter arranged through the access sheath 400 and carrying a constricted LAAO device with the left atrial appendage.

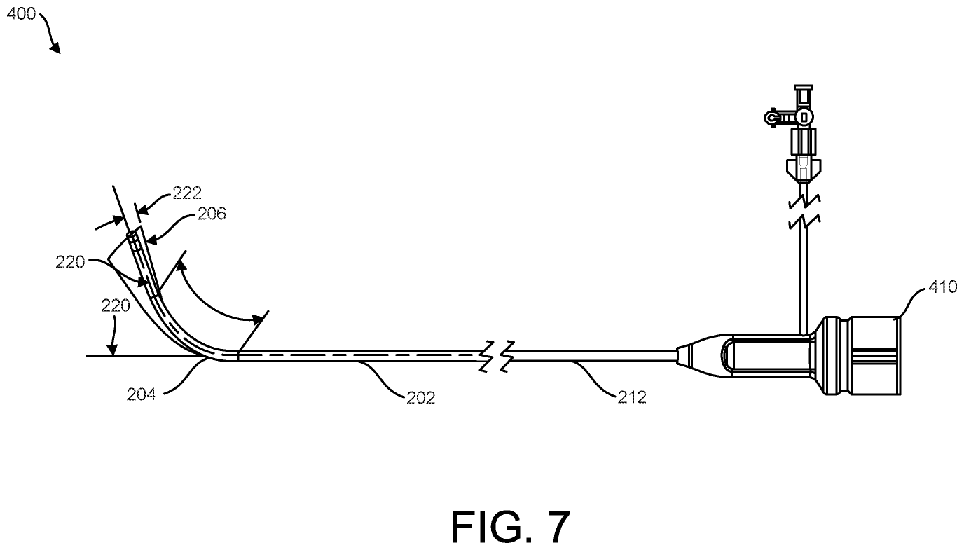

FIG. 7 is a side view of the access sheath 400, shown in FIGS. 2-6, in accordance with various aspects of the present disclosure. As shown in FIG. 7, the distal end 416 of the access sheath 400 includes the two curvatures discussed in detail above. The proximal curved segment 204 defines a proximal nominal angular offset 220 and the distal curved segment 206 extends defines a distal nominal angular offset 222.

In certain instances, the proximal nominal angular offset 220 is between approximately 65 and 75 degrees and the distal nominal angular offset 222 is between approximately 25 and 35 degrees. In other instances, the proximal nominal angular offset 220 is approximately 70 degrees and the distal nominal angular offset 22 is approximately 30 degrees. In certain instances, these curvatures are in the planes noted above relative to FIGS. 2-6. For example, the proximal curved segment 204 and the distal curved segment 206 are in different planes and offset between approximately 80-110 degrees.

As shown in FIG. 7, the proximal curved segment 204 and the distal curved segment 206 are curved in a common direction relative to the plane shown. The access sheath 400 may include a projection indicating a direction of curvature of the proximal curved segment 204. In certain instances, a port 410 coupled to the proximal portion 212 of the elongate body 202 with the port 410 extending in a direction relative to the elongate body 202 in a common direction with the proximal curved segment 204.

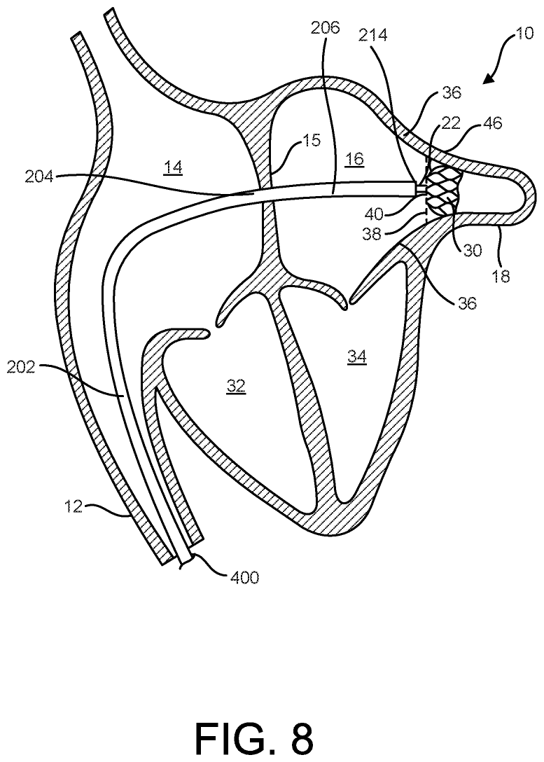

FIG. 8 is a cross-sectional view of a human heart 10 in which an access sheath 400 is positioned in preparation for deployment of an implantable device 30 into an appendage 18 of the heart, in accordance with various aspects of the present disclosure. The depiction includes a right atrium 14, a left atrium 16, a right ventricle 32, and a left ventricle 34 of the heart 10. As is shown, the appendage 18 is located in the left atrium 16 of the heart 10, and thus, the appendage 18 may be considered the left atrial appendage 18. Although the following discussion focuses on deployment of the implantable device 30 into the left atrial appendage 18, the implantable device 30 may be deployed in other appendages or openings within the human heart 10 or in other locations of the human body.

The left atrial appendage 18 may be considered a muscular pouch extending from the anterolateral wall 36 of the left atrium 16 of the heart 10, which serves as a reservoir for the left atrium 16. In a normal cardiac cycle, the left atrial appendage 18 may contract rhythmically with the rest of the left atrium 16 during contraction of the heart 10. Thus, during a normal cardiac cycle, the left atrial appendage 18 contracts with the left atrium 16 and pumps blood that may gather or collect within the left atrial appendage 18 to circulate therefrom. However, during cardiac cycles characterized by arrhythmias (e.g., atrial fibrillation), the left atrial appendage 18 may fail to sufficiently contract along with the left atrium 16, which can allow blood to stagnate within the left atrial appendage 18. Stagnant blood within the atrial appendage 18 is susceptible to coagulating and forming a thrombus, which can dislodge from the atrial appendage 18 and ultimately result in an embolic stroke. The implantable device 30, consistent with various aspects of the present disclosure, may be delivered to the left atrial appendage 18 to help prevent and militate against blood stagnation within the left atrial appendage 18.

In certain instances and as is shown in FIG. 8, the implantable device 30 may be delivered to the left atrial appendage 18 by way of a minimally invasive transcatheter procedure. More specifically, the access sheath 400 may be navigated through a vena cava 12, into the right atrium 14, through an atrial septum 15, and into the left atrium 16 towards the appendage 18. In some implementations, the percutaneous access to the patient's vasculature can be at the patient's femoral vein, for example. It should be understood that this example technique is merely one example, and many other access techniques can also be performed to deploy the occlusive devices provided herein. At this point of the deployment process, the occlusive device is contained within a lumen of the access sheath 400, and is configured in a collapsed low-profile delivery configuration. Although transcatheter systems are generally shown and described, other delivery systems (e.g., thoracoscopic) are also contemplated.

As shown, a delivery catheter 22 may releasably couple to the implantable device 30, and is slidably disposed within the lumen of the access sheath 400. The delivery catheter 22 can be used by a clinician operator to make the implantable device 30 deploy from the access sheath 400. For example, after positioning the implantable device 30 through an ostium 38 of the left atrial appendage 18, the clinician operator unsheathes and deploys the implantable device 30.

The access sheath 400, as discussed in further detail above, facilitates delivery of the device 300 to a target location within a patient. A distal portion 416 of the access sheath 400 has a distal opening 216 and, as discussed above, curved segments 204, 206 that are configured to orient the distal opening 216 relative to the target location (in this case, the left atrial appendage 18). In certain instances and is shown, the device 30 is an atrial appendage occluder and the proximal curved segment 204 is configured to guide the elongate body 202 through a septum 15 in the heart 10 and turn toward the left atrial appendage 18. Further and in certain instances, the distal curved segment 206 is configured to align the catheter 22 with a longitudinal axis of the left atrial appendage 18.

In certain instances, the delivery catheter 22 is configured to pass through the internal lumen of the elongate body 202 and the elongate body 202 is configured to maintain the curvatures (e.g., proximal nominal angular offset and the distal nominal angular offset) of the proximal curved segment 204 and the distal curved segment 206 when the catheter 22 is within a distal portion of the elongate body 202.

The access sheath 400 may be shaped or curved depending on the device used for treatment or diagnosis. The access sheath 400 may be used for other implantable medical devices such as occluder, stents, stent-grafts, shunts, and can also be used for ablation catheters, sensors, pressure/diagnostics devices, drug delivery systems, contrast solution delivery, or other similar devices.

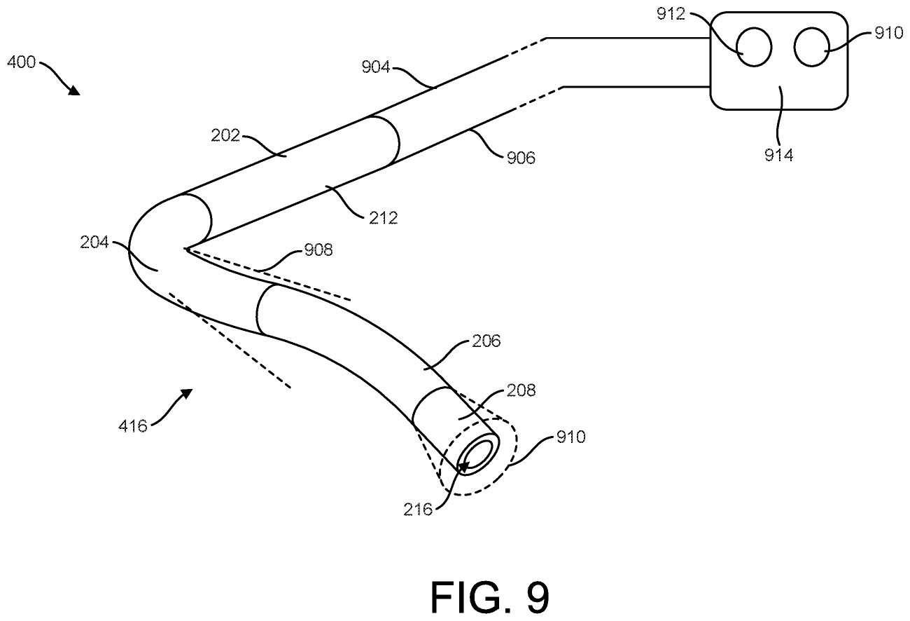

FIG. 9 is a perspective view of an example access sheath 400 that may be steerable, in accordance with various aspects of the present disclosure. The access sheath 400 may include one or more steering features to alter a shape of the elongate body 202. The access sheath 400, as shown, may include a distal portion 416 having a plurality of segments that are configured to orient a distal opening 216 of the access sheath 400 relative to the target location. In certain instances, one or more of the segments may include a curvature. The amount and number of curvatures can depend on the use of the access sheath 400 (e.g., aortic device delivery or diagnostics including into the ascending aorta, descending aorta, and abdominal aorta; trans-septal delivery of devices or diagnostics; septal occluder delivery; septal occluder delivery with venous access; mitral valve repair; aortic valve repair; left atrial appendage occlusion; other device delivery, repair, or diagnostics of the left ventricle or atrium; and other similar applications).

In certain instances, the access sheath 400 may include a proximal steering element 904 configured to actuate a proximal one of the plurality of segments of the distal portion 416. As shown, the access sheath 400 includes a proximal curved segment 204, and the proximal steering element is configured to actuate the proximal curved segment 204 of the access sheath 400. In certain instances, the proximal steering element 904 configured to actuate the proximal curved segment 204 of the access sheath 204 in-plane 908. The proximal steering element 904 may change an amount or radius of curvature of the proximal curved segment 204.

In certain instances, the access sheath 400 may include a distal steering element 906 configured to actuate a distal one of the plurality of segments of the distal portion 416. As shown, the access sheath 400 includes a distal curved segment 206. In certain instances, the distal steering element 906 is configured to actuate the distal curved segment 206 of the access sheath 400.

In certain instances, the distal steering element 906 is configured to actuate the distal curved segment 206 of the access sheath 204 to rotate the distal curved segment 206. The distal steering element 906 may rotate the distal curved segment 206 in within a 360 degree angulation (e.g., a cone) to alter the direction the end of the elongate body 202 is directing, for example.

In certain instances, the distal steering element 906 is configured to deflect the distal curved segment 206 about a 360 degree range. For example, the distal steering element 906 may be configured to deflect the distal curved segment 206 to positions about the 360 degree range.

The proximal steering element 904 and the distal steering element 906 may be embedded in the access sheath 400 and coupled, respectively, to the proximal curved segment 204 and the distal curved segment 206.

In certain instances, the access sheath 400 includes no steering mechanism to alter curvature (e.g., the curved segments 204, 206 are fixed). In other instances, the access sheath 400 can include only a proximal steering element 904, only a distal steering element 906, or both a proximal steering element 904 and a distal steering element 906. In certain instances, the initial curvatures of the proximal curved segment 204 and/or the distal curved segment 206 facilitate a starting point for steering and limit the amount of steering that is possible by the proximal steering element 904 and the distal steering element 906.

In certain instances, the proximal steering element 904 is a wire or fiber that alters the curvature of the proximal curved segment 204 in response to tension. The proximal steering element 904 may be bidirectional such that the application of a force in a direction opposite or toward the physician alters a curvature of the proximal curved segment 204.

In certain instances, the distal steering element 906 is a wire or fiber that rotates the distal curved segment 206 in response to tension. The distal steering element 906 may be bidirectional such that the application of a force in a direction opposite or toward the physician alters a rotation of the distal curved segment 206.

In these instances, the proximal steering element 904 may be bidirectional such that the application of a force in a direction opposite or toward the physician alters the pre-set curvature of the proximal curved segment 204 within a range of approximately −180° to +180°, typically in the range of −90° to +90°, possibly in the range of −60° to +60°, −45° to +45°, −30° to +30° or less within a plane in which the curve is set relative to the pre-set amount of curvature as describe above with reference to FIGS. 2-6. Reference may be made to U.S. Pat. No. 7,666,204 (“Thorton et al.”) and WO 2019/023477 (“Prabhu et al.”) for further discussion of pull wires manipulating a pre-set curved portion of a catheter.

In certain instances, the steering elements 904, 906 may include articulating structures (e.g., a series or balloons or bladders) that are arranged in selected portions of the access sheath 400 to effect curvature or rotation of the curved segments 204, 206. The articulating structures (e.g., a series or balloons or bladders) may be individually actuated to effect curvature or rotation of the curved segments 204, 206. Reference may be made to U.S. Pat. No. 4,982,165 (“Loiterman”), U.S. Patent Publication 2016/0279388 (“Barrish et al.”), and/or WO 2018/200738 (“Laby et al.”) for further discussion of articulating structures.

The steering elements 904, 906 can be coupled to a handle 914 arranged at or near a proximal end of the access sheath 400. The handle 914 may include steering structures 910, 912 that are coupled to the steering elements 904, 906 that may actuate the steering elements 904, 906 in response user intervention. The steering structures 910, 912 may be knobs, dials, levers, or other similar structures.

In certain instances, the steering elements 904, 906 may be configured to steer the access sheath 400 to particular offsets or curvatures. For example, the proximal steering element 904 may be able to deflect a segment of the distal portion 416 (e.g., a proximal one of the plurality of segments noted above) to extend within in a first plane to define a proximal nominal angular offset in the direction of extension of the elongate body 202. In addition, the distal steering element 906 may be configured to deflect a segment of the distal portion 416 (e.g., a distal one of the plurality of segments noted above) within a second plane to define a distal nominal angular offset in the direction of extension of the elongate body 202.

The distal portion 416 may begin with curves with the steering elements 904, 906 being configured to alter the curvatures of segments of the distal portion 416. The steering elements 904, 906 may also be configured to deflect segments of the distal portion 416 to be within a desired curvature. For example, the steering elements 904, 906 may be configured to deflect the distal portion 416 to have the proximal nominal angular approximately 25 and 35 degrees. In addition, the steering elements 904, 906 may be configured to deflect the distal portion 416 to have the proximal nominal angular offset is approximately 70 degrees and the distal nominal angular offset is approximately 30 degrees.

The curved segments 204, 206 may begin with a curvature such that the steering elements 904, 906 are configured to deflect the proximal curved segment 204 and the distal curved segment 206 to different planes offset between approximately 80-110 degrees (e.g., as shown in FIGS. 2-7). In certain instances, the curved segments 204, 206 may begin with a curvature that is within 0-5%, 5-10%, 10-15%, 15-20%, 20-25%, 25-30%, 30-40%, 40-50%, 50-60%, 60-70%, 70-80%, 80-90%, 90-99% or any range therebetween of the final deflection range that, the steering elements 904, 906 are configured to provide. Thus, the steering elements 904, 906 may be configured to further deflect the curved segments 204, 206 from the beginning curvature to deflect the proximal curved segment 204 and the distal curved segment 206 to different planes offset between approximately 80-110 degrees (e.g., as shown in FIGS. 2-7).

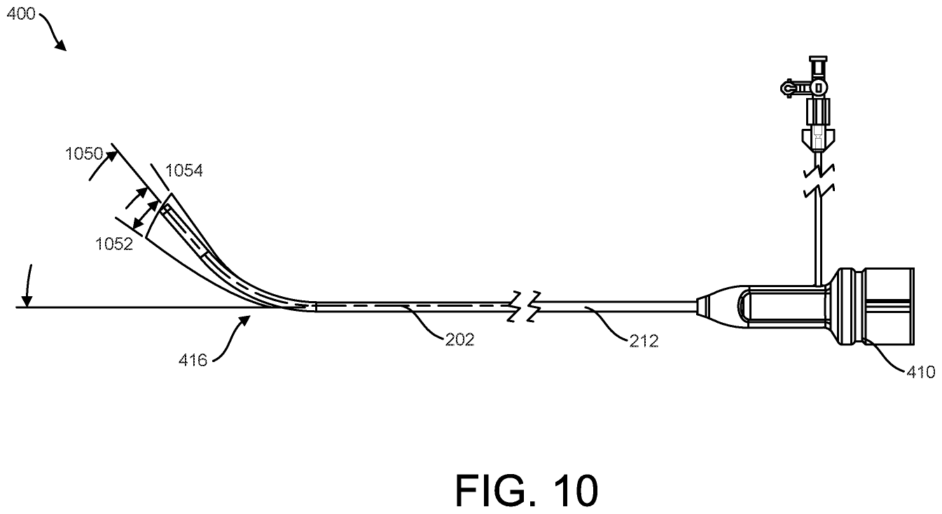

FIG. 10 is side view of an another example access sheath 400 in accordance with various aspects of the present disclosure. As shown in FIG. 10, the distal portion 416 of the access sheath 400 includes a single curvature in the plane shown. The access sheath 400 may include a proximal portion 212 that is at least partially accessible outside of a patient. The proximal portion 212 extends in a direction of extension, which may be a first plane. In certain instances, a port 410 coupled to a proximal portion 212 of the elongate body 202 with the port 410 extending in a direction relative to the elongate body 202 in a common direction with the distal portion 416.

In certain instances, the distal portion 416 may be gradually curved. The distal portion 416 may include a total curvature 1050, relative to the proximal portion 212, of approximately 40-70 degrees. In certain instances, the total curvature 1050 may be approximately 65 degrees (+/−5%). In certain instances, the distal portion 416 may include a first curvature 1052 of 5-15 degrees relative to the proximal portion 212 on an outside curve of the elongate body 202. In certain instances, the first curvature 1052 may be approximately 10 degrees (+/−5%). In certain instances, the distal portion 416 may include a second curvature 1054 of 5-15 degrees relative to the proximal portion 212 on an inside curve of the elongate body 202. In certain instances, the second curvature 1054 may be approximately 10 degrees (+/−5%).

The distal portion 416 may be deflected, relative to the total curvature 1050, by using one or both steering elements 904, 906 described in detail with reference to FIG. 9. The steering elements 912, 914 may also be configured to deflect the distal portion 416 to include one or more additional curvatures than the single curvature shown and or alternatively the steering elements 912, 914 may be configured to deflect the distal portion 416 into different planes.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

What is claimed is:

1. A delivery system comprising:
an access sheath including an elongate body with an internal lumen to facilitate delivery of a device to a target location within a patient, the elongate body extending in a direction of extension and including:
a proximal portion extending in a first plane, and
a distal portion having a distal opening and a plurality of curved segments configured to orient the distal opening relative to the target location, the distal portion including,
a proximal curved segment extending within in a first plane to define a proximal nominal angular offset in the direction of extension of the elongate body, and
a distal curved segment extending within a second plane to define a distal nominal angular offset in the direction of extension of the elongate body, the first and second planes being angularly offset from one another.

2. The delivery system of claim 1, wherein the proximal nominal angular offset is between approximately 65 and 75 degrees and the distal nominal angular offset is between approximately 25 and 35 degrees.

3. The delivery system of claim 2, wherein the proximal nominal angular offset is approximately 70 degrees and the distal nominal angular offset is approximately 30 degrees.

4. The delivery system of claim 1, wherein the proximal curved segment and the distal curved segment are in different planes and offset between approximately 80-110 degrees.

5. The delivery system of claim 1, wherein the proximal curved segment is an x-z plane the distal curved segment is in a y-z plane.

6. The delivery system of claim 1, further including a catheter configured to pass through the internal lumen of the elongate body and the elongate body is configured to maintain the proximal nominal angular offset and the distal nominal angular offset when the catheter is within the distal portion of the elongate body.

7. The delivery system of claim 6, wherein the catheter facilitates delivery of an implantable medical device.

8. The delivery system of claim 7, wherein the implantable medical device is selected from a group consisting of: a left atrial appendage occluder, an occluder, a stent, a stent-graft, a shunt, a sensor, a pressure sensing device, and a diagnostic device.

9. The delivery system of claim 7, wherein the implantable medical device is a left atrial appendage occluder and the proximal curved segment is configured to guide the elongate body through a septum in heart turn toward a left atrial appendage and the distal curved segment is configured to align the catheter with a longitudinal axis of the left atrial appendage.

10. The delivery system of claim 6, wherein the catheter is selected from a group consisting of a medical device delivery catheter, an ablation catheter a drug delivery catheter, and a contrast solution delivery catheter.

11. The delivery system of claim 1, further comprising a projection coupled to the proximal portion of the elongate body configured to indicate a direction of the proximal curved segment.

12. The delivery system of claim 11, wherein the projection is a port coupled to the proximal portion of the elongate body, and the port extends in a direction relative to the elongate body in a common direction as the proximal curved segment.

13. The delivery system of claim 1, further comprising a proximal steering element configured to actuate the proximal curved segment in-plane.

14. The delivery system of claim 13, wherein the proximal steering element is a steering wire coupled to the proximal curved segment configured to actuate the proximal curved segment in response to a force applied to the steering wire.

15. The delivery system of claim 13, wherein the proximal steering element includes a series of articulating structures arranged about the proximal curved segment and configured to individually actuate to effect curvature of the proximal curved segment.

16. The delivery system of claim 1, further comprising a distal steering element configured to rotate the distal curved segment.

17. The delivery system of claim 16, wherein the distal steering element is a steering wire coupled to the distal curved segment configured to rotate the distal curved segment in response to a force applied to the steering wire.

18. The delivery system of claim 1, further comprising a distal steering element configured to deflect the distal curved segment about a 360 degree range.

19. The delivery system of claim 18, wherein the distal steering element is a steering wire coupled to the distal curved segment configured to deflect the distal curved segment in response to a force applied to the steering wire.

20. The delivery system of claim 18, wherein the distal steering element includes a series of articulating structures arranged about the distal curved segment and configured to individually actuate to effect deflection of the distal curved segment.

21. The delivery system of claim 13, wherein distal steering element includes a series of articulating structures arranged about the distal curved segment and configured to individually actuate to effect rotation of the distal curved segment.

22. The delivery system of claim 13, wherein the access sheath includes the proximal steering element and the distal steering element.

23. A method of delivering an implantable medical device to a target location within a patient, the method comprising:
arranging an access sheath within a patient, the access sheath including an elongate body with an internal lumen, a proximal portion extending in a first plane, and a distal portion having a distal opening and a plurality of curved segments; and
orienting the distal opening of the access sheath relative to the target location using a proximal curved segment of the distal portion extending within in a first plane to define a proximal nominal angular offset in the direction of extension of the elongate body and a distal curved segment of the distal portion extending within a second plane to define a distal nominal angular offset in the direction of extension of the elongate body, the first and second planes being angularly offset from one another.

24. The method of claim 23, further comprising arranging a delivery catheter through the internal lumen of the access sheath and delivering an implantable medical device arranged on the delivery catheter through the distal opening of the access sheath to the target location.

25. The method of claim 24, wherein the implantable medical device is a left atrial appendage occluder and the proximal curved segment is configured to guide the elongate body through a septum in heart turn toward a left atrial appendage and the distal curved segment is configured to align the catheter with a longitudinal axis of the left atrial appendage.

26. A delivery system comprising:
an access sheath including an elongate body with an internal lumen to facilitate delivery of a device to a target location within a patient, the elongate body extending in a direction of extension and including:
a proximal portion extending in a first plane, and
a distal portion having a distal opening and a plurality of segments configured to orient the distal opening relative to the target location,
a proximal steering element configured to deflect a proximal one of the plurality of segments to extend within in a first plane to define a proximal nominal angular offset in the direction of extension of the elongate body, and
a distal steering element configured to deflect a distal one of the plurality of segments within a second plane to define a distal nominal angular offset in the direction of extension of the elongate body.

27. The delivery system of claim 26, wherein the proximal nominal angular offset is between approximately 65 and 75 degrees and the distal nominal angular offset is between approximately 25 and 35 degrees.

28. The delivery system of claim 27, wherein the proximal nominal angular offset is approximately 70 degrees and the distal nominal angular offset is approximately 30 degrees.

29. The delivery system of claim 26, wherein at least one of the proximal one of the plurality of segments and the distal one of the plurality of segments is substantially aligned with the direction of extension of the elongate body prior to deflection.

30. The delivery system of claim 26, wherein the proximal one of the plurality of segments is a proximal curved segment prior to deflection.

31. The delivery system of claim 30, wherein the distal one of the plurality of segments is a distal curved segment prior to deflection.

32. The delivery system of claim 31, wherein the proximal steering element and the distal steering element and configured to deflect the proximal curved segment and the distal curved segment to different planes offset between approximately 80-110 degrees.

33. The delivery system of claim 26, wherein the first and second planes being angularly offset from one another.
